(19) United States
(12) Patent Application Publication
(10) Pub. No.: US 2025/0249060 A1
(43) Pub. Date: Aug. 7, 2025

(54) APPLICATION METHOD OF ACTIVE PORTION OF LIGUSTICUM CHUANXIONG IN PREPARATION OF ANTI-GOUT DRUG

(71) Applicant: Shaanxi University of Chinese Medicine, Xianyang (CN)

(72) Inventors: Zhi-Shu Tang, Xianyang (CN); Hong-Bo Xu, Xianyang (CN); Rui Zhou, Xianyang (CN); Xiao-Hui Hu, Xianyang (CN); Kang Ma, Xianyang (CN)

(21) Appl. No.: 18/431,986

(22) Filed: Feb. 4, 2024

(51) Int. Cl. A61K 36/236; A61P 19/06

(52) U.S. Cl. CPC A61K 36/236; A61P 19/06

(57) ABSTRACT

An application method of an active portion of Ligusticum chuanxiong in preparation of an anti-gout drug is provided, which belongs to the technical field of drugs, relates to an active portion of a traditional Chinese medicine and an application method thereof. An active portion of the Ligusticum chuanxiong for inhibiting xanthine oxidase (XOD, or XO) is an ethyl acetate extract, the ethyl acetate extract mainly includes a small and medium polar lipophilic component, and the small and medium polar lipophilic component is mainly a phthalide compound, and it has been confirmed through experiments that the active portion has an anti-gout effect, in addition, an anti-gout drug and an anti-gout health product containing the active portion of the Ligusticum chuanxiong are provided.

### TECHNICAL FIELD

The disclosure belongs to the technical field of drugs, relates to an active portion of a traditional Chinese medicine and an application method thereof, and more particularly to an application method of an active portion of Ligusticum chuanxiong in preparation of an anti-gout drug.

### BACKGROUND

A traditional Chinese medicine Ligusticum chuanxiong is a dry rhizome of Ligusticum chuanxiong Hort. “Chinese Pharmacopoeia” records that the Ligusticum chuanxiong has functions in “promoting blood circulation and activating qi, and dispelling pathogenic wind and relieving pain”, and the Ligusticum chuanxiong is mainly used to treat chest and hypochondriac pain, swelling pain due to stumbling, amenorrhea and dysmenorrhea, pain of gynecologic abdominal lumps, headaches, rheumatic disorder and other diseases. Modern pharmacological studies show that the Ligusticum chuanxiong has various pharmacologic action, such as vasodilation, antiplatelet aggregation and anti-thrombosis, antioxidant, neuroprotection, anti-inflammation, antibiosis, liver and kidney protection, sedation, asthma relief, anti-proliferation, and pro-apoptosis.

Gout is a common inflammatory arthropathy induced by long-term purine metabolism disorder, increased blood uric acid levels, and deposition of monosodium urate crystals (MSU) in joints. Xanthine oxidase (XOD, or XO) in a human body can convert hypoxanthine to xanthine, and then convert the xanthine to uric acid. Therefore, the XOD is a most commonly used target for modern medical treatment of the gout. XOD inhibitor can reduce generation of the uric acid, thus having an anti-gout effect. Up to now, there are only two such drugs on the market worldwide, namely allopurinol and febuxostat.

According to understanding of traditional Chinese medicine (TCM) on the gout, the TCM classifies the gout as “arthralgia syndrome”, when treating the gout, Chinese medicines with functions of dispelling pathogenic wind, relieving pain and activating blood circulation are adopted in conjunction with characteristics of disease progression. Therefore, the Ligusticum chuanxiong is often used in a compound compatibility for the gout in the clinical practice of TCM, and the compound compatibility includes Sangdang decoction, Tong-bi decoction and the like.

However, up to now, there is no systematic study and research on anti-gout effect, active substances, active portion, possible main components, and the underlying mechanism of the Ligusticum chuanxiong at home and abroad. In 1998, Korean scholars have studied an inhibitory effect of 80% methanol extract of the Ligusticum chuanxiong on the XOD in vitro, and found that the inhibitory effect is weak. In 2017, the inventor discovers that an extract obtained from the Ligusticum chuanxiong after methanol extraction has a significant inhibitory effect on the XOD in vitro, and a half maximal inhibitory concentration (IC₅₀) of the extract is 11.8 micrograms per millimeter (μg/mL), which has an equivalent effect compared to positive control allopurinol (IC₅₀=6.4 μg/mL). In further in-depth research, the inventor obtains an active portion of the Ligusticum chuanxiong for inhibiting the XOD, namely ethyl acetate portion, and the ethyl acetate extract with the anti-gout effect is preliminary proven in vivo. Meanwhile, main components of the active portion are characterized by using a liquid chromatography mass spectrometry (LC-MS) technology, the result shows that it mainly includes a lipophilic (small and medium polarity) component, and the lipophilic component is mainly phthalide compound.

In summary, the inventor first identified the anti-gout active portion of the Ligusticum chuanxiong, and preliminarily revealed that the active portion may play an anti-gout role by inhibiting the XOD, and the active portion can be used as an alternative material for preparing a drug or a health product for preventing and treating the gout.

### SUMMARY

A purpose of the disclosure is to provide an application method of an active portion of Ligusticum chuanxiong in preparation of an anti-gout drug.

In order to achieve the above purpose, the disclosure discloses a preparation method, main components, and pharmacological effects of the active portion in vivo and in vitro.

On the one hand, the disclosure relates to the application method of the active portion of the Ligusticum chuanxiong in preparation of the anti-gout drug, and discloses that the active portion of the Ligusticum chuanxiong is an ethyl acetate extract, and a half maximal inhibitory concentration (IC₅₀) of the ethyl acetate extract for inhibiting xanthine oxidase (XOD, or XO) is in a range of 18.25±0.19 (i.e., 18.06-18.44) micrograms per millimeter (μg/mL).

Specifically, the active portion of the Ligusticum chuanxiong (i.e., the ethyl acetate extract) is obtained by using ethyl acetate to extract a Ligusticum chuanxiong solution. The Ligusticum chuanxiong solution is a Ligusticum chuanxiong aqueous solution or a Ligusticum chuanxiong alcohol solution.

Specifically, the disclosure identifies the active portion of the Ligusticum chuanxiong, that is, the ethyl acetate extract of the Ligusticum chuanxiong mainly includes a small and medium polar lipophilic component, and the small and medium polar lipophilic component is mainly a phthalide compound.

On the other hand, the disclosure relates to an anti-gout drug or an anti-gout health product, and the anti-gout drug or the anti-gout health product includes the ethyl acetate extract of the Ligusticum chuanxiong.

The active portion of the Ligusticum chuanxiong described in the disclosure can be prepared into any convenient dosage forms suitable for use selected from the group consisting of but not limited to a tablet, a pill, a paste, a spray, a capsule and the like. Furthermore, in order to fully utilize pharmacological effects of the drugs provided by the disclosure, when preparing the convenient dosage forms of the drug, it is necessary to add not only necessary active component, the active portion of the Ligusticum chuanxiong, but also an assistant or a carrier to assist effective component of the active component to exert the pharmacological effects, the assistant or the carrier includes at least one of flavoring agent, disintegrating agent, cosolvent and buffering agent, which are commonly used assistants of the drug or the health product. The disclosure does not specifically limit the assistant or the carrier, and they can be selected or adjusted according to actual application.

Compared to the related art, the disclosure has the following beneficial effects or advantages.
(1) As a natural compound derived from Chinese herbal medicine, the active portion of the Ligusticum chuanxiong has advantages of environmental friendliness, low drug resistance, less environmental residue, low biological toxicity and natural degradation, which can effectively relieve drug residue, environmental pollution and drug resistance caused by chemicals and antibiotics, and is a green and pollution-free aquatic product.
(2) An inventor identifies that a key substance basis of the Ligusticum chuanxiong for inhibiting the XOD is the ethyl acetate extract, the IC₅₀ value of the ethyl acetate extract for inhibiting the XOD is in a range of 18.25±0.19 μg/mL, and the ethyl acetate extract can significantly reduce activity of the XOD in serum and liver of a mouse, significantly reduce a uric acid content in serum of a mouse in a hyperuricemia model, significantly relieve a feet swelling degree of a mouse with acute gouty arthritis, and has no significant effect in weight of the mouse.

### BRIEF DESCRIPTION OF DRAWINGS

FIG. 1 illustrates a schematic diagram of inhibition rates of extracts/liquid-liquid extracts of Ligusticum chuanxiong on xanthine oxidase (XOD) according to an embodiment of the disclosure. Specifically, A: allopurinol; B: water extract of the Ligusticum chuanxiong; C: methanol extract of the Ligusticum chuanxiong; D: ethyl acetate extract of the Ligusticum chuanxiong; E: residual of ethyl acetate liquid-liquid extract of the Ligusticum chuanxiong; F: ethyl acetate liquid-liquid extract of the Ligusticum chuanxiong.

FIG. 2 illustrates a schematic curve diagram of an inhibitory effect of the ethyl acetate extract of the Ligusticum chuanxiong on the XOD according to an embodiment of the disclosure.

FIG. 3 illustrates a schematic diagram of an effect of the ethyl acetate extract of the Ligusticum chuanxiong on serum XOD activity in hyperuricemia mice according to an embodiment of the disclosure; specifically, compared to a normal control group, ##P<0.01; and compared to a model group, ***P<0.001, and ****P<0.0001.

FIG. 4 illustrates a schematic diagram of an effect of the ethyl acetate extract of the Ligusticum chuanxiong on the activity of liver XOD in hyperuricemia mice according to an embodiment of the disclosure; specifically, compared to the normal control group, ##P<0.01; and compared to the model group, ***P<0.001.

FIG. 5 illustrates a schematic diagram of an effect of the ethyl acetate extract of the Ligusticum chuanxiong on the serum uric acid in hyperuricemia mice according to an embodiment of the disclosure; specifically, compared to the normal control group, ###P<0.001; and compared to the model group, ***P<0.001, and ****P<0.001.

FIG. 6 illustrates a schematic diagram of an effect of the ethyl acetate extract of the Ligusticum chuanxiong on the paw swelling in gouty arthritis mice according to an embodiment of the disclosure; specifically, compared to a normal control group, ###P<0.001; and compared to a model group, **P<0.01, and ***P<0.001.

FIG. 7 illustrates a schematic diagram of an effect of the ethyl acetate extract of the Ligusticum chuanxiong on body weight in gouty arthritis mice according to an embodiment of the disclosure.

FIG. 8 illustrates a schematic diagram of an effect of the ethyl acetate extract of the Ligusticum chuanxiong on the activity of serum XOD in hyperuricemia rats according to an embodiment of the disclosure; specifically, compared to a normal control group, ###P<0.001; and compared to a model group, **P<0.01, and ***P<0.001.

FIG. 9 illustrates a schematic diagram of an effect of the ethyl acetate extract of the Ligusticum chuanxiong on the serum uric acid in hyperuricemia rats according to an embodiment of the disclosure; specifically, compared to the normal control group, ###P<0.001; and compared to the model group, *P<0.05, and ***P<0.001.

FIG. 10 illustrates a schematic diagram of an effect of the ethyl acetate extract of the Ligusticum chuanxiong on the serum urea nitrogen in hyperuricemia rats according to an embodiment of the disclosure; specifically, compared to the normal control group, ###P<0.001; and compared to the model group, **P<0.01, and ***P<0.001.

FIG. 11 illustrates a schematic diagram of an effect of the ethyl acetate extract of the Ligusticum chuanxiong on the serum creatinine in hyperuricemia rats according to an embodiment of the disclosure; specifically, compared to the normal control group, ###P<0.001; and compared to the model group, P<0.001.

FIG. 12 illustrates a schematic diagram of an effect of the ethyl acetate extract of the Ligusticum chuanxiong on the kidney in hyperuricemia rats according to an embodiment of the disclosure.

FIG. 13 illustrates a schematic diagram of an effect of the ethyl acetate extract of the Ligusticum chuanxiong on the pain in gouty arthritis rats according to an embodiment of the disclosure; specifically, compared to a normal control group, ###P<0.001; and compared to a model group, ***P<0.001.

FIG. 14 illustrates a schematic diagram of an effect of the ethyl acetate extract of the Ligusticum chuanxiong on the joint morphology in gouty arthritis rats according to an embodiment of the disclosure.

FIG. 15 illustrates a schematic diagram of an effect of the ethyl acetate extract of the Ligusticum chuanxiong on the serum levels of interleukin-1 beta (IL-1) in gouty arthritis rats according to an embodiment of the disclosure; specifically, compared to the normal control group, #P<0.05; and compared to the model group, *P<0.05.

FIG. 16 illustrates a schematic diagram of an effect of the ethyl acetate extract of the Ligusticum chuanxiong on the serum levels of tumor necrosis factor-α (TNF-α) in gouty arthritis rats according to an embodiment of the disclosure; specifically, compared to the normal control group, #P<0.05; and compared to the model group, *P<0.05, and **P<0.01.

FIG. 17 illustrates a schematic diagram of an effect of the ethyl acetate extract of the Ligusticum chuanxiong on the pathological changes of joint synovitis (also referred to as arthrosynovitis) in gouty arthritis rats according to an embodiment of the disclosure.

FIG. 18A illustrates a schematic diagram of an ultra-performance liquid chromatography (UPLC) spectrum of the ethyl acetate extract of the Ligusticum chuanxiong according to an embodiment of the disclosure.

FIG. 18B illustrates a schematic diagram of a total ion chromatogram (TIC) of the ethyl acetate extract of the Ligusticum chuanxiong according to an embodiment of the disclosure.

FIG. 19 illustrates a schematic diagram of structures of main components in the ethyl acetate extract of the Ligusticum chuanxiong according to an embodiment of the disclosure.

### DETAILED DESCRIPTION OF EMBODIMENTS

Technical solutions of the disclosure are described in conjunction with embodiments below, however, the disclosure is not limited to the following embodiments.

Unless otherwise specified, experimental methods and detection methods in the following embodiments are all conventional methods; unless otherwise specified, medicines and materials can be purchased in a market; and unless otherwise specified, index data are all obtained through conventional measurement methods.

### Embodiment 1

The embodiment provides a confirmation of an active portion of Ligusticum chuanxiong.

Xanthine oxidase (XOD, or XO) for clinical prevention and treatment of gout is taken as a breakthrough point, based on a previous research foundation, that is, the inventor discovers that a methanol extract of the Ligusticum chuanxiong has a significant inhibitory effect on the XOD in vitro, in order to further confirm the active portion of the Ligusticum chuanxiong for inhibiting the XOD, the inventor designs the following experiments.

1. Preparations of Extracts/Liquid-Liquid Extracts of the Ligusticum chuanxiong
4 parts of Ligusticum chuanxiong decoction pieces are weighed, each part of the Ligusticum chuanxiong decoction pieces is 50 grams (g), and these decoction pieces are processed as follows.

For the first sample, 200 milliliters (mL) of water is added to 50 g decoction pieces to obtain a first mixture, and ultrasonic extraction is performed twice on the first mixture for 1 hour (h) each time to obtain first extraction solutions. After combining and concentrating the first extraction solutions to dry, a water extract of Ligusticum chuanxiong (B) is obtained.

For the second sample, 200 mL of methanol is added to 50 g decoction pieces to obtain a second mixture, and ultrasonic extraction is performed twice on the second mixture for 1 h each time to obtain second extraction solutions. After combining and concentrating second extraction solutions to dry, a methanol extract of Ligusticum chuanxiong (C) is obtained.

For the third sample, 200 mL of ethyl acetate is added to 50 g decoction pieces to obtain a third mixture, and ultrasonic extraction is performed twice on the third mixture for 1 h each time to obtain third extraction solutions. After combining and concentrating third extraction solutions to dry, an ethyl acetate extract of Ligusticum chuanxiong (D) is obtained.

For the fourth sample, 200 mL of methanol is added to 50 g decoction pieces to obtain a fourth mixture, and ultrasonic extraction is performed twice on the fourth mixture for 1 h each time to obtain fourth extraction solutions. After combining and concentrating the fourth extraction solutions to dry to obtain a dried mixture. Then the dried mixture is dispersed in 150 ml of water, and liquid-liquid extracted three times with equal amount of the ethyl acetate to obtain aqueous solution and three liquid-liquid extracts, the three liquid-liquid extracts are combined to obtain an ethyl acetate liquid-liquid extract solution. After that the aqueous solution and the ethyl acetate liquid-liquid extract solution are concentrated to dry respectively, and a residual of ethyl acetate liquid-liquid extract (E) and an ethyl acetate liquid-liquid extract (F) are obtained.

2. The Inhibitory Effect of the Extracts/Liquid-Liquid Extracts of the Ligusticum chuanxiong on the Activity of XOD.

2.1 Buffer solution preparation: 0.4780 g of potassium phosphate monobasic (KH₂PO₄), 3.473 g of dipotassium hydrogen phosphate trihydrate (K₂HPO₄·3H₂O) and 10.00 milligrams (mg) of ethylenediaminetetraacetic acid disodium salt (EDTA-2Na) are accurately weighed, and then placed in a 250 mL volumetric flask, and diluted to volume with distilled water to obtain the buffer solution.

2.2 Substrate preparation: 3.65 mg of xanthine is accurately weighed, and then added into the buffer solution to dilute to volume of 50 mL, and the xanthine is dissolved with ultrasound to obtain the substrate.

2.3 Enzyme solution preparation: XOD (5 units abbreviated as U) is taken and diluted with 50 mL of the buffer solution to obtain a diluted solution, and the diluted solution is subpackaged in sample bottles to obtain the enzyme solution, and kept in a temperature of −70 Celsius degree (° C.) for later use.

2.4 A positive control sample preparation: 2 mg of allopurinol is weighed, 1 mL of methyl sulfoxide (DMSO) and 8 mL of phosphate buffered saline (PBS) buffer solution are added to obtain a mixture, and the ultrasound is performed on the mixture for promoting dissolution of the allopurinol.

2.5 Tested samples preparation: 1.0 mg of samples B, C, D, E, F are respectively weighed, each sample is dissolved in 10 microliters (L) of DMSO with ultrasound to obtain a solution, the buffer solution is added into the solution to a volume of the solution be 1 mL, and a solution with a concentration of 1 milligram of milliliter (mg/mL) is obtained. After that the solution is filtered with a microporous filter membrane to obtain the samples to be tested for later use.

2.6 XOD activity determination: A 96-well plate is taken, 100 μL of each tested sample and 100 μL of the positive control sample are respectively sucked, 10 μL of DMSO and 90 μL of the buffer solution are sucked as a blank group. 40 μL of the enzyme solution is added into each well, after incubating the 96-well plate in a microplate reader at a temperature of 37° C. for 3 minutes (min), 60 μL of the substrate (a total volume of the substrate is 200 μL) is added into the 96-well plate, and an absorbance value is immediately measured once every 30 seconds (s) at 295 nanometers (nm) for a total of 15 min.

2.7 A calculation formula of an inhibition rate (%) of the XOD: Inhibition rate (%)=[1−(ΔA sample/ΔA blank)]×100%, and ΔA is a difference of absorbance at a certain time.

2.8 A half maximal inhibitory concentration (IC₅₀) value measurement: the active portion is prepared into 7 series concentrations to explore a dose-effect relationship based on results of the activity test, and IC₅₀ value of the active portion is calculated through a GraphPad prism 8 software.

### 3. Results and Analysis.

3.1 Confirmation of the active portion of the Ligusticum chuanxiong for inhibiting the XOD

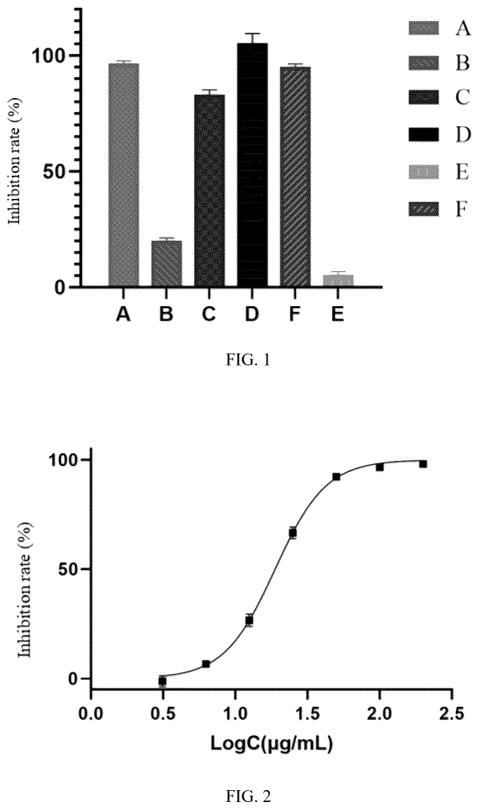

The inhibitory effect of the positive control sample, the allopurinol (A), and the extracts/the liquid-liquid extracts of the Ligusticum chuanxiong (B-F) on the XOD are shown in FIG. 1. The methanol extract (83.14%) and the ethyl acetate extract (105.38%) of the Ligusticum chuanxiong have good inhibition rates on activity of the XOD, and the ethyl acetate extract of the Ligusticum chuanxiong has a stronger inhibition rate on the activity of the XOD, which shows a trend of complete inhibition similar to the positive control sample, allopurinol (96.78%). When the water, the methanol and the ethyl acetate are respectively used as solvents to extract the Ligusticum chuanxiong, there is a phenomenon of component overlap in obtained extracts, especially the methanol extract contains a lot of the same components as the ethyl acetate extract. The methanol extract of the Ligusticum chuanxiong is divided into the ethyl acetate liquid-liquid extract (F) and a residual of the ethyl acetate liquid-liquid extract (E) by the inventor, and inhibition rates of the ethyl acetate liquid-liquid extract (F) and the residual of the ethyl acetate liquid-liquid extract (E) on the activity of the XOD are respectively measured. Results show that an inhibitory effect of E on the XOD almost disappears, and the inhibition rate is only 5.31%. In this situation, F shows a strong inhibitory effect (an inhibition rate is 95.25%), and the inhibitory effect of F is roughly equivalent to that of D. In summary, it can be seen that the ethyl acetate extract/liquid-liquid extract of the Ligusticum chuanxiong is the active portion of the Ligusticum chuanxiong for inhibiting the XOD in vitro.

3.2 Determination of an IC₅₀ value of XOD inhibition by the ethyl acetate extract of the Ligusticum chuanxiong

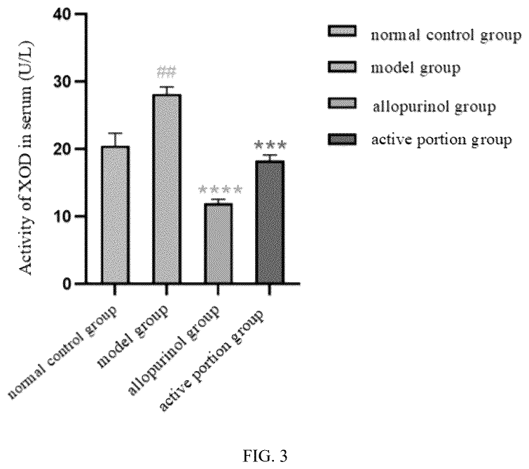

Inhibition rates of series concentration of the ethyl acetate extract of the Ligusticum chuanxiong on the XOD is measured according to a measurement method of the activity of the XOD to draw an activity curve, the activity curve is shown in FIG. 2, and the IC₅₀ values of XOD inhibition by the ethyl acetate extract of the Ligusticum chuanxiong is 18.25±0.19 μg/mL.

### Embodiment 2

The embodiment provides in vivo experimental validation of an anti-gout arthritis effect of the ethyl acetate extract of the Ligusticum chuanxiong in mice.

### 1. Experimental Materials

1.1 Kunming male mice, 18-20 g, purchased from Chengdu Dossy Experimental Animals CO., LTD., and a license key is [SCXK(Chuan)2020-030].

1.2 Experimental reagents include the follows. Potassium oxonate (PO) and monosodium urate (MSU) are purchased from Sigma-Aldrich (Shanghai) Trading Co., Ltd.; physiological saline with a concentration of 0.9% are purchased from Xian Jingxi Double-Crane Pharmaceutical Co., Ltd.; sodium carboxymethyl cellulose and colchicine pieces are purchased from Guangdong Pidi Pharmaceutical Co., LTD.; and iodine, ethanol, pentobarbital sodium, uric acid assay kit and xanthine oxidase (XOD, or XO) kit are purchased from Nanjing Jiancheng Technology Co., Ltd.

1.3 Experimental instruments include the follows. A multiskan spectrum is purchased from Thermo Fisher Scientific Company; a UV-2600 UV-Vis spectrophotometer is purchased from Shimadzu Company, Kyoto, Japan; a vortex mixer (a model number is VORTEX-6) is purchased from Hangzhou Xuqing Technology Co., Ltd.; an ice machine (a model number is AC-270) is purchased from Beijing Kangjian Meibo Technology Development Co., Ltd.; a one-hundred-thousandth electronic balance (a model number is CPA225D) is purchased from Sartorius Scientific Instruments Co., Ltd.; and a paw volume measuring instrument (a model number is YSL-7C) is purchased from Shandong Academy Of Medical Sciences Jinan Yiyan Technology Development Co., Ltd.

### 2. Experimental Method

2.1 Preparation of a sodium carboxymethyl cellulose suspension is as follows. 800 mL of ultrapure water is heated to a temperature of 70° C., and placed on a magnetic stirrer, 4 g of sodium carboxymethyl cellulose is poured into the ultrapure water slowly and stirred for 2 h to obtain a clarified colloidal liquid with no visible lumps, the clarified colloidal liquid is placed in a refrigerator at 4° C. overnight to obtain 5% sodium carboxymethyl cellulose suspension.

2.2 Preparation of a test sample (i.e., the ethyl acetate extract of the Ligusticum chuanxiong) is as follows. 1 g of the ethyl acetate extract of the Ligusticum chuanxiong is dissolved 20 mL of 0.5% sodium carboxymethyl cellulose suspension, and the ultrasound is performed to promote dissolution of the ethyl acetate extract of the Ligusticum chuanxiong to obtain 50 mg/mL suspension of the ethyl acetate extract of the Ligusticum chuanxiong.

2.3 Preparation of a first positive control sample (allopurinol) is as follows. 0.04 g of allopurinol is dissolved in 10 mL of the sodium carboxymethyl cellulose suspension with a concentration of 0.5% to obtain an allopurinol suspension.

2.4 Preparation of a second positive control sample (colchicine) is as follows. 0.5 mg of colchicine is dissolved in 5 mL of the sodium carboxymethyl cellulose suspension with a concentration of 0.5% to obtain a colchicine suspension with a concentration of 0.1 mg/mL.

2.5 Preparation of PO is as follows. 4 g of PO salt is grinded and then added into 0.5% sodium carboxymethyl cellulose suspension slowly to obtain a mixture, the mixture is repeatedly blown and beaten until there are no visible particles to naked eye to obtain a white suspension (PO suspension).

2.6 Preparation of MSU crystal is as follows. 400 mg of sodium urate is added into 77 mL of the ultrapure water, 2.5 mL of sodium hydroxide (NaOH) with a concentration of 1 molar per liter (μmol/L) is added and heated together with the sodium urate and the ultrapure water to a temperature of 95° C. to completely dissolve the sodium urate, to thereby obtain a mixed solution. After cooling, a potential of hydrogen (pH) of the mixed solution is adjusted to 7.0 with hydrochloric acid (HCl), and then the mixed solution is placed in the refrigerator with a temperature of 4° C. for 24 h to precipitate a crystal. The mixed solution with the crystal is centrifuged with a speed of 3000 revolutions per minute (r/min) for 2 min, with a total of 3 times, until no more crystals precipitate. The obtained crystals are dried at a temperature of 100° C. for 2 h to obtain dried crystals for later use. When using the dried crystals, the PBS buffer solution is added to prepare a MSU crystal suspension with a concentration of 20 mg/mL.

2.7 Grouping and administration of the experimental animals

After the male Kunming mice are adaptively fed, the male Kunming mice are randomly divided into a normal control group of mice, a model group of mice with hyperuricemia, an allopurinol group of mice with hyperuricemia, an ethyl acetate extract of the Ligusticum chuanxiong group of mice with hyperuricemia, a normal control group of mice, a model group of mice with acute gouty arthritis, a colchicine group of mice with acute gouty arthritis and an ethyl acetate extract of the Ligusticum chuanxiong group of mice with acute gouty arthritis, and each group has 8 male Kunming mice. The allopurinol group of mice with hyperuricemia is daily administered the allopurinol suspension by gavage, the colchicine group of mice with acute gouty arthritis is daily administered the colchicine suspension by gavage, the ethyl acetate extract of the Ligusticum chuanxiong group of mice with hyperuricemia and the ethyl acetate extract of the Ligusticum chuanxiong group of mice with acute gouty arthritis are daily administered the suspension of the ethyl acetate extract of the Ligusticum chuanxiong by gavage, the normal control group of mice, the normal control group of mice, the model group of mice with hyperuricemia and the model group of mice with acute gouty arthritis are daily administered the sodium carboxymethyl cellulose suspension with a concentration of 0.5% by gavage, administration volumes are 10 milliliters per kilogram (mL/kg), and it is given continuously for 7 days.

1 hour before the last administration, the model group of mice with hyperuricemia, the allopurinol group of mice with hyperuricemia and the ethyl acetate extract of the Ligusticum chuanxiong group of mice with hyperuricemia are intraperitoneally injected the PO suspension with administration volumes of 280 milligrams per kilogram (mg/kg). 1 h after administration, blood of all mice is taken through retrobulbar venous plexus, the blood is allowed to stand at a room temperature for 30 min, and then is centrifuged with a speed of 3000 r/min to obtain serum of the blood, after repeating twice, the serum of the mice is collected. Livers of the mice are taken and kept in a temperature of −80° C. for later use. The normal control group of mice is injected with 0.1 mL of physiological saline onto right foot pad of the mice after 1 h gavage on the 7^{th} day. The model group of mice with acute gouty arthritis, the colchicine group of mice with acute gouty arthritis and the ethyl acetate extract of the Ligusticum chuanxiong group of mice with acute gouty arthritis are injected with 0.1 mL of the MSU crystal suspension onto right foot pad of the mice.

### 3. Index Detection

3.1 Detection of the activity of the XOD in hyperuricemia mice

Physiological saline with a concentration of 0.9% is added into the livers of the mice according to a ratio of 1:9, and liver homogenate is prepared by using a mechanical homogenization method. The liver homogenate is centrifuged with a speed of 6000 r/min to sperate supernatant for later use. The activity of the XOD in the blood and the liver are evaluated through a colorimetric determination of enzyme activity.

3.2 Measurement of uric acid contents in the serum of hyperuricemia mice

The uric acid contents are measured through a colorimetry, and a construction effect of a hyperuricemia model and an effect of the ethyl acetate extract of the Ligusticum chuanxiong on the uric acid of the mice in the hyperuricemia model are evaluated.

3.3 Measurement of paw swelling degrees in hyperuricemia mice

0 h, 4 h, 8 h, 12 h and 24 h after the MSU crystal suspension injection, paw volumes of the mice are measured by using the paw volume measuring instrument, the paw swelling degrees of the mice are calculated, and the calculation formula is as follows:

$$paw swelling degree ( \% ) = ( {C}_{t} - {C}_{0} ) / {C}_{0} ;$$

where Cₜ represents a paw volume at different time points after injection, and Co represents a paw volume at 0 h after injection.

3.4 A statistics method

The GraphPad Prism 8 software is adopted to process data. The data are represented by using average ±standard error (i.e., x̅±SEM), differences between different treatments are statistically analyzed by a t-test and a one-way analysis of variance (ANOVA), and checked by a Bonferroni correction method.

### 4. Results and Analysis

4.1 The effect of the ethyl acetate extract of the Ligusticum chuanxiong on the activity of the serum XOD in hyperuricemia mice

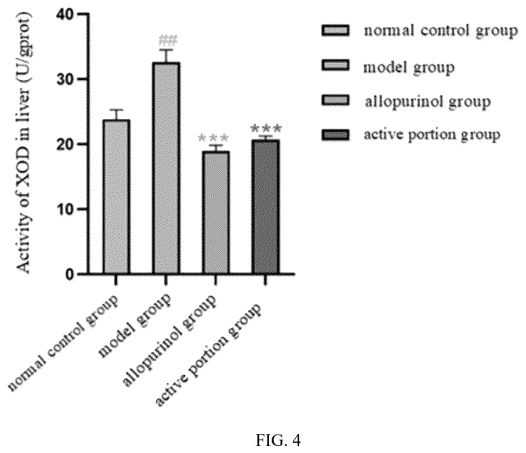

As shown in FIG. 3, in hyperuricemia mice, the activity of the serum XOD of the model group is 28.19 units per liter (U/L), which increased by 37.32% compared to the normal control group (20.53 U/L), with a statistical difference (P<0.01), and the result indicates a successful construction of the model. Compared to the model group, the activity of the serum XOD of the allopurinol group is 11.99 U/L, which decreased by 57.45% (P<0.0001). Similar to the allopurinol group, the activity of the serum XOD of the ethyl acetate extract of the Ligusticum chuanxiong group is 18.32 U/L, which decreased by 34.99% (P<0.001). The result indicates that the ethyl acetate extract of the Ligusticum chuanxiong has a significant inhibitory effect on the activity of the serum XOD in hyperuricemia mice.

4.2 The effect of the ethyl acetate extract of the Ligusticum chuanxiong on the activity of the XOD in the livers of hyperuricemia mice

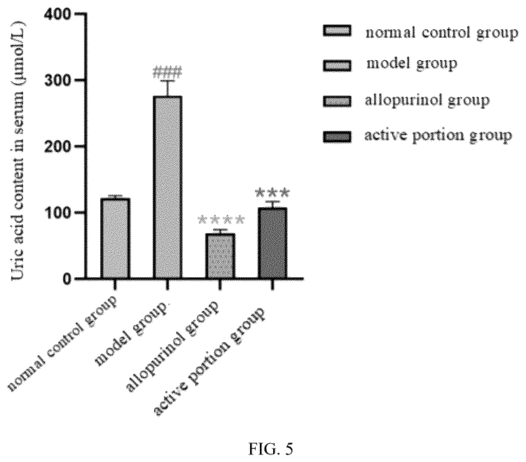

As sown in FIG. 4, in hyperuricemia mice, the activity of the liver XOD of the model group is 32.62 units per g protein (U/gprot), which increased by 36.83% compared to the normal control group (23.85 U/L), and the result indicates a successful construction of the model. Compared to the model group, the activity of the liver XOD of the allopurinol group is 18.95 U/gprot, which decreased by 41.92% (P<0.001). Similar to the allopurinol group of mice, the activity of the liver XOD of the ethyl acetate extract of the Ligusticum chuanxiong group is 20.74 U/gprot, which decreased by 36.44% (P<0.05). The result indicates that the ethyl acetate extract of the Ligusticum chuanxiong has a significant inhibitory effect on the activity of the liver XOD of hyperuricemia mice.

4.3 The effect of the ethyl acetate extract of the Ligusticum chuanxiong on the serum uric acid level in hyperuricemia mice

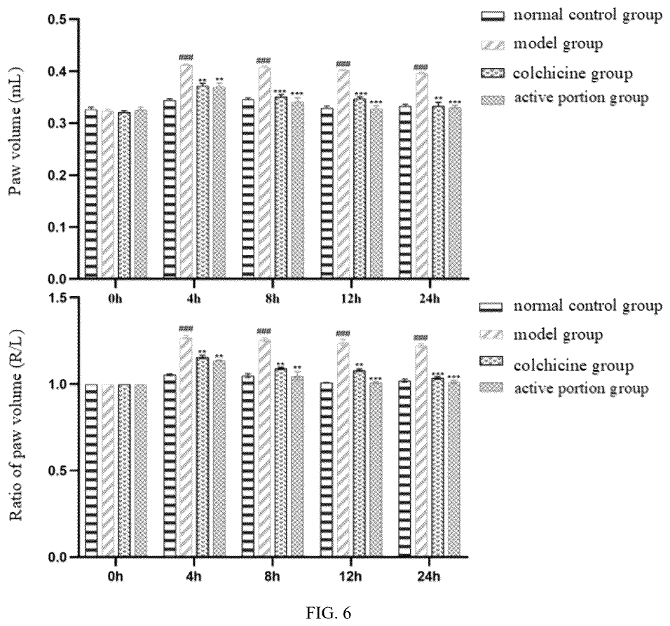

As shown in FIG. 5, in hyperuricemia mice, the serum uric acid level is 286.82 micromolar per liter (μmol/L), compared to the normal control group, the serum uric acid level increased by 75.16%, with a statistical difference (P<0.001), and the result indicates a successful construction of the model. Compared to the model group, the serum uric acid level of the allopurinol group is 68.72 μmol/L, which decreased by 75.16% (P<0.001). Similar to the allopurinol group, the serum uric acid level of the ethyl acetate extract of the Ligusticum chuanxiong group is 108.14 μmol/L, which decreased by 60.92% (P<0.001). The result indicates that the ethyl acetate extract of the Ligusticum chuanxiong can significantly decrease the serum uric acid level in hyperuricemia mice.

4.4 The effect of the ethyl acetate extract of the Ligusticum chuanxiong on the paw swelling degrees of acute gouty arthritis in mice

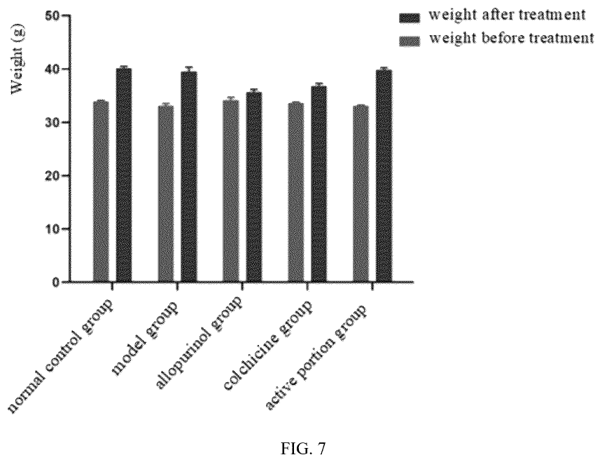

As shown in FIG. 6, after injecting the MSU crystal suspension into the foot pads of the mice for 0 h, 4 h, 8 h, 12 h, and 24 h, compared to the normal control group (injection with the physiological saline), the paw swelling degrees in the model group increased over 19% (P<0.0001), which indicates that a mouse model with acute gouty arthritis is successfully constructed, and the model is stable during research.

In acute gouty arthritis mice, the colchicine shows a strong inhibitory effect at 4 h, 8 h, 12 h and 24 h, the paw swelling degree is decreased more than 9% (P<0.001) compared with the model group, and reached the highest (15.32%) at the 24 h. Meanwhile, the ethyl acetate extract of the Ligusticum chuanxiong also shows a similar inhibitory effect on the paw swelling degree at the corresponding time, compared with the model group, the inhibitory effects of the ethyl acetate extract of the Ligusticum chuanxiong on the paw swelling degree at 4 h, 8 h, 12 h and 24 h are decreased by 10.51%, 16.68%, 18.78% and 17.09% respectively, and the inhibitory effect on the paw swelling is even better than that of the colchicine group.

4.5 The effect of the ethyl acetate extract of the Ligusticum chuanxiong on the weight of acute gouty arthritis in mice

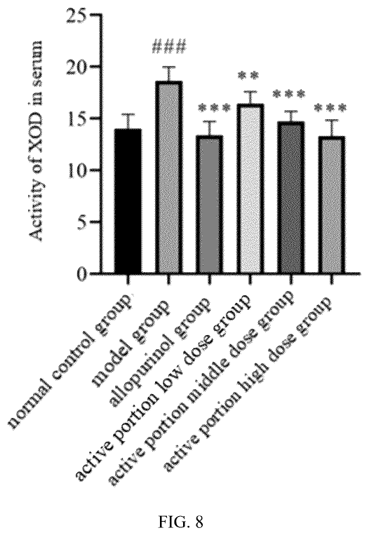

As shown in FIG. 7, compared to the normal control group, weight gain in the allopurinol group and the colchicine group are significantly lower, which indicate that the allopurinol (40 milligrams per kilogram per day abbreviated as mg/kg·d) and the colchicine (1 mg/kg·d) have a possible effect on weight gain of the mice. Weight gain in the ethyl acetate extract of the Ligusticum chuanxiong group (500 mg/kg·d) before and after treatment are similar to that of the normal control group, which indicate that the ethyl acetate extract of the Ligusticum chuanxiong group has no effect on weight gain of the mice.

### Embodiment 3

The embodiment provides in vivo validation of the ethyl acetate extract of the Ligusticum chuanxiong against hyperuricemia in rats.

### 1. Experimental Materials

1.1 Experimental animals are 72 male specific pathogen-free (SPF) level Sprague-Dawley (SD) rats with 7 weeks old and weight of 220±20 g, which are purchased from the Chengdu Dossy Experimental Animals CO., LTD., a production license number for the experimental animals is [SCXK(Chuan) 2021-036], the animal experiment has been approved by Experimental Animal Ethics Committee of Shaanxi University of Traditional Chinese Medicine, and an ethical code is SUCMDL20230206002.

1.2 Experimental reagents include the follows. Ligusticum chuanxiong decoction pieces is purchased from Shaanxi Sciendan Pharmaceutical Co., Ltd., and a lot number is 20211203; PO, hypoxanthine, and sodium carboxymethyl cellulose (CMC) are purchased from Shanghai Yuanye Bio-Technology Co., Ltd., and lot numbers are 517112-100 g, 518025-100 g and Y25A10X86590; a uric acid test kit, a urea nitrogen test kit, and a XOD kits are purchased from Nanjing Jiancheng Technology Co., Ltd., and lot numbers are C012-2-01, C013-2-1 and A002-1-1; and allopurinol pieces are purchased from Heifei Jiulian Pharma Co., Ltd., and a lot number is 20220301.

1.3 Experimental instruments include the follows. A Thermo an GO 1510 multiskan spectrum, a Micro17R micro freezing centrifuge, a Thermo Scientific Forma 900 Series −80° C. ultra-low temperature refrigerator are purchased from Thermo Fisher Scientific Company; and an IX73 invert microscope is purchased from Olympus Microoptical Technology Company.

### 2. Experimental Method

2.1 Preparation of a sodium carboxymethyl cellulose suspension: 800 mL of ultrapure water is heated to a temperature of 70° C., and placed on the magnetic stirrer, 4 g of sodium carboxymethyl cellulose is poured into the ultrapure water slowly and stirred for 2 h to obtain a clarified colloidal liquid with no visible lumps, the clarified colloidal liquid is placed in a refrigerator at 4° C. overnight to obtain a sodium carboxymethyl cellulose suspension with a concentration of 0.5%.

2.2 Preparation of active portion (i.e., the ethyl acetate extract of the Ligusticum chuanxiong): 32.4 mg, 64.8 mg and 129.6 mg of the ethyl acetate extract of the Ligusticum chuanxiong are dissolved in 20 mL of the sodium carboxymethyl cellulose suspension with a concentration of 0.5% respectively, and dissolved with the ultrasound to obtain a suspension of the ethyl acetate extract of the Ligusticum chuanxiong with a concentration of 1.62 mg/mL, 3.24 mg/mL, and 6.48 mg/mL.

2.3 Preparation of a positive control sample (i.e., the allopurinol): 1.2 mg of the allopurinol is dissolved in 40 mL of the sodium carboxymethyl cellulose suspension with a concentration of 0.5% to obtain an allopurinol suspension with a concentration of 0.3 mg/mL.

2.4 Preparation of PO: 1.2 g of PO salt is added into 12 mL of physiological saline slowly to obtain a first mixture, the first mixture is repeatedly blown and beaten until there are no visible particles to naked eye to obtain a white suspension (i.e., PO suspension) with a concentration of 50 mg/mL.

2.5 Preparation of hypoxanthine: 6 g of the PO salt is added into 120 mL of 0.5% sodium carboxymethyl cellulose suspension slowly to obtain a second mixture, the second mixture is blown repeatedly and beaten until there are no visible particles to the naked eye to obtain a white suspension (i.e., a hypoxanthine suspension) with a concentration of 50 mg/mL.

### 3. Index Detection

3.1 Detection of the activity of the serum XODin hyperuricemia rats

The activity of the serum XOD of hyperuricemia in rats is measured by using the colorimetry, and a hyperuricemia model and the effect of the ethyl acetate extract of the Ligusticum chuanxiong on the activity of the serum XOD of hyperuricemia in rats are evaluated.

3.2 Determination of the serum uric acid in hyperuricemia rats

The serum uric acid of the rats with hyperuricemia is measured by using the colorimetry, and the hyperuricemia model and the effect of the ethyl acetate extract of the Ligusticum chuanxiong on the serum uric acid of the rats with hyperuricemia are evaluated.

3.3 Detection of serum urea nitrogen in hyperuricemia rats

The serum urea nitrogen of the rat with hyperuricemia is measured by using the colorimetry, and the hyperuricemia model and the effect of the ethyl acetate extract of the Ligusticum chuanxiong on the serum urea nitrogen of the rats with hyperuricemia are evaluated.

3.4 Detection of serum creatinine in hyperuricemia rats

The serum creatinine of the rats with hyperuricemia is measured by using the colorimetry, and the hyperuricemia model and the effect of the ethyl acetate extract of the Ligusticum chuanxiong on the serum creatinine of the rats with hyperuricemia are evaluated.

3.5 Kidneys pathological examination

Kidney tissues of the rats are cleaned to obtain cleaned kidney tissues, the cleaned kidney tissues are placed in a sufficient 4% paraformaldehyde fixative for fixing at least 24 h. A paraffin embedding is performed on the cleaned kidney tissues to obtain a kidney tissue paraffin, the kidney tissue paraffin is sliced with a slicing machine to obtain paraffin sections. The paraffin sections are individually dewaxed with xylene I and xylene II for 15 min to obtain dewaxed sections, and the dewaxed sections are individually rehydrated with an ethanol gradient from high to low to obtain rehydrated sections. The rehydrated sections are placed into a hematoxylin dye solution for 3-5 min, followed by washing with purified water, differentiating with a differentiation solution, rinsing with pure water, staining with eosin for 4 min, returning to blue with a bluing reagent, dehydrating with the ethanol gradient from high to low after rinsing, using xylene to be transparent, and sealing with a neutral balsam to obtain sealed sections. In this case, pathological changes of kidneys in the rats are observed under a light microscope. Microscopic examination, and image acquisition and analysis are performed.

### 4. Results and Analysis

4.1 The effect of the ethyl acetate extract of the Ligusticum chuanxiong on the activity of the serum XOD in hyperuricemia in rats

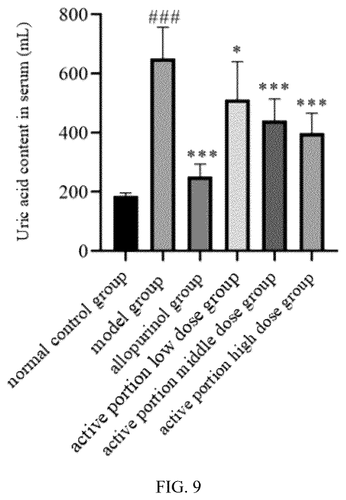

As shown in FIG. 8, the activity of the serum XOD of a model group is 30 U/L, which increased significantly compared to the normal control group (18 U/L), with a statistical difference (P<0.001). Compared to the model group, the serum XOD activity in the allopurinol group significantly decreases, and the serum XOD activity in the ethyl acetate extract of the Ligusticum chuanxiong group also significantly decreases, which indicates that the ethyl acetate extract of the Ligusticum chuanxiong has a significant inhibitory effect on the serum XOD activity in hyperuricemia rats.

4.2 The effect of the ethyl acetate extract of the Ligusticum chuanxiong on the serum uric acid in hyperuricemia in rats

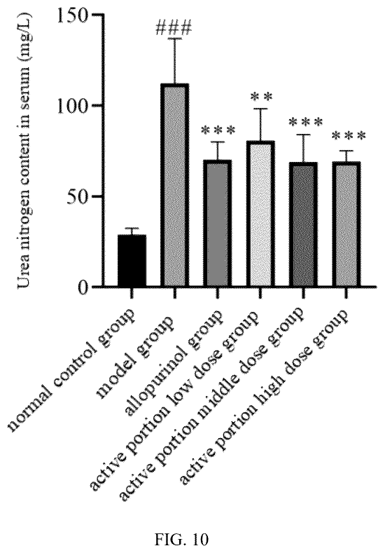

As shown in FIG. 9, compared to the normal control group, the serum uric acid level in the model group increases significantly, the ethyl acetate extract of the Ligusticum chuanxiong can obviously reduce the level of the serum uric acid. The result demonstrates that the inhibitory effect of the ethyl acetate extract of the Ligusticum chuanxiong on the serum uric acid in hyperuricemia rats.

4.3 The effect of the ethyl acetate extract of the Ligusticum chuanxiong on the serum urea nitrogen in hyperuricemia rats

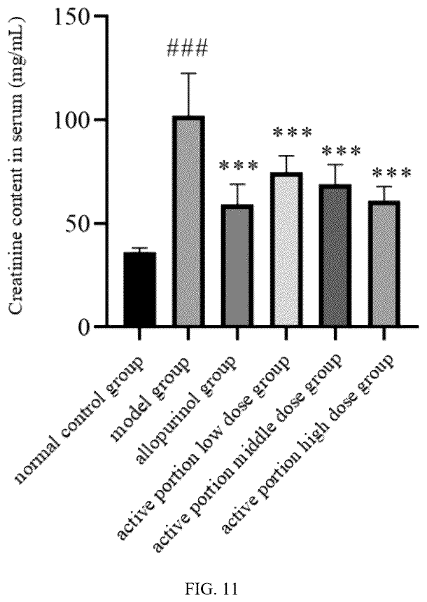

As shown in FIG. 10, compared to the normal control group, the serum urea nitrogen level in the model group increases significantly, the ethyl acetate extract of the Ligusticum chuanxiong can obviously reduce generation of the serum urea nitrogen. The result demonstrates that the inhibitory effect of the ethyl acetate extract of the Ligusticum chuanxiong on the serum urea nitrogen in hyperuricemia rats.

4.4 The effect of the ethyl acetate extract of the Ligusticum chuanxiong on the serum creatinine in hyperuricemia trats

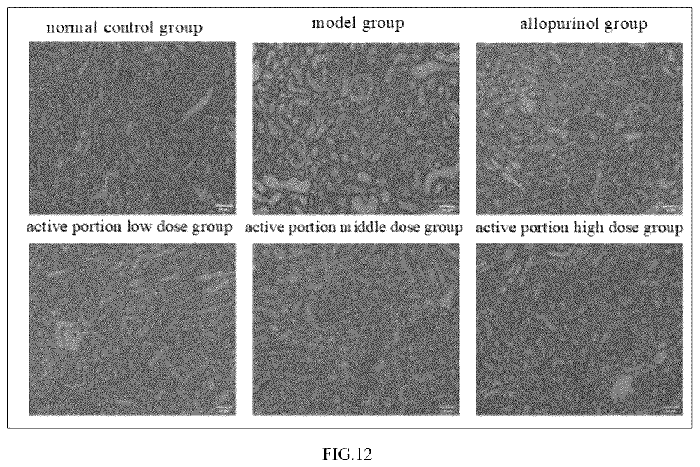

As shown in FIG. 11, compared to the normal control group, the serum creatinine level in the model group increases significantly, the ethyl acetate extract of the Ligusticum chuanxiong can obviously reduce the generation of the serum creatinine. The result demonstrates that the inhibitory effect of the ethyl acetate extract of the Ligusticum chuanxiong on the serum creatinine in hyperuricemia rats.

4.5 The effect of the ethyl acetate extract of the Ligusticum chuanxiong on the pathological of kidneys in hyperuricemia rats

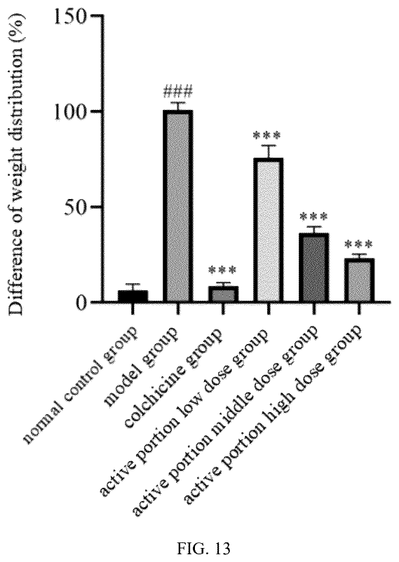

As shown in FIG. 12, a hematoxylin-eosin (HE) staining is performed on the kidneys of the rats with hyperuricemia in the research, compared to the normal control group, aggregated secretions in renal tubular lumen of the rats in the model group are increased, color is light, and the renal tubular lumen is obviously dilated with local edema. However, after treatment with the ethyl acetate extract of the Ligusticum chuanxiong, the renal tubular lumen is dilated and edema is decreased. The results demonstrate that the ethyl acetate extract of the Ligusticum chuanxiong treatment obviously improve kidneys injury in hyperuricemia rats.

4.6 A statistical method

All experiments are performed individually, and each experiment is repeated at least three times. Data is represented by average ±standard deviation. A SPSS 20.0 software and a GraphPad Prism 9.0 software are adopted to analyze the data, the one-way analysis of variance is used for inter group analysis, and a Tukey test is used for paired comparison. P<0.05 has statistical significance.

### Embodiment 4

The embodiment provides in vivo validation of the ethyl acetate extract of the Ligusticum chuanxiong against an acute gouty arthritis in rats.

### 1. Experimental Materials

1.1 36 male SPF level SD rats with 7 weeks old and weight of 220±20 g is purchased from the Chengdu Dossy Experimental Animals CO., LTD., a production license number for the experimental animals is [SCXK(Chuan) 2021-036], the animal experiment has been approved by the Experimental Animal Ethics Committee of Shaanxi University of Traditional Chinese Medicine, and an ethical code is SUCMDL202214005.

1.2 Experimental reagents include the follows. MSU is purchased from Sigma-Aldrich (Shanghai) trading Co., Ltd.; physiological saline with a concentration of 0.9% is purchased from Xian Jingxi Double-Crane Pharmaceutical Co., Ltd.; sodium carboxymethyl cellulose and colchicine are purchased from Yunnan Phytopharmaceutical Co., LTD., and a lot number is 20210514; an interleukin 1 beta (IL-1β) and tumor necrosis factor-α enzyme-linked immunosorbent assay (TNF-α ELISA) kit of rat are purchased from Neobioscience, and lot numbers are R221208-007a and R221219-102a; anhydrous ethanol, xylene and neutral gum are purchased from Sinopharm Chemical Reagent CO., LTD., and lot numbers are 100092683, 10023418 and 10004160; and a HE dye solution is purchased from Wuhan Servicebio Technology Co., Ltd., and a lot number is G1003.

1.3 Experimental instruments include the follows. A KW-11A bipedal balance pain meter is purchased from Nanjing Calvin Biotechnology Co., Ltd.; a Thermo an GO 1510 multiskan spectrum, a Micro17R micro freezing centrifuge, a Thermo Scientific Forma 900 Series −80° C. ultra-low temperature refrigerator are purchased from Thermo Fisher Scientific Company; and an IX73 invert microscope is purchased from Olympus Microoptical Technology Company; a ME204/02 electronic balance is purchased from Mettler-Toledo Co., Ltd.; a YSL-7C paw volume measuring instrument is purchased from Jinan Yiyan Science & Technology Development Co., Ltd.; a Leica CM1860UV freezing microtome is purchased from Leica Biosysterms Nussloch GmbH Company; an EYELA N-1300 rotary evaporator is purchased from Tokyo Physical and Chemical Instruments Corporation; a KQ-300 DE type ultrasonic cleaner is purchased from Kunshan Ultrasonic Instruments CO., LTD.; and a UXFSTPRP-64 high speed centrifugal grinder is purchased from Eppendorf Company.

### 2. Experimental Method

2.1 Preparation of a sodium carboxymethyl cellulose suspension: 800 mL of ultrapure water is heated to a temperature of 70° C., and placed on a magnetic stirrer, 4 g of sodium carboxymethyl cellulose is poured into the ultrapure water slowly and stirred for 2 h to obtain a clarified colloidal liquid with no visible lumps, the clarified colloidal liquid is placed in a refrigerator at 4° C. overnight to obtain a sodium carboxymethyl cellulose suspension with a concentration of 0.5%.

2.2 Preparation of an active portion (i.e., the ethyl acetate extract of the Ligusticum chuanxiong): 32.4 mg, 64.8 mg and 129.6 mg of the ethyl acetate extract of the Ligusticum chuanxiong are dissolved in 20 mL of 0.5% sodium carboxymethyl cellulose suspension respectively, and dissolved with the ultrasound to obtain suspensions of the ethyl acetate extract of the Ligusticum chuanxiong with concentrations of 1.62 mg/mL, 3.24 mg/mL, and 6.48 mg/mL.

2.3 Preparation of a positive control sample (i.e., the colchicine): 0.9 mg of the colchicine is dissolved in 30 mL of 0.5% sodium carboxymethyl cellulose suspension to obtain a colchicine suspension with a concentration of 0.3 mg/mL.

2.4 Preparation of a MSU crystal suspension: 400 mg of sodium urate is added into 70 mL of ultrapure water, 2.5 mL of NaOH with a concentration of 1 μmol/L is added and heated together with the sodium urate and the ultrapure water to a temperature of 95° C. to dissolve the sodium urate completely, to thereby obtain a mixed solution. After cooling, a pH of the mixed solution is adjusted to 7.0 with hydrochloric acid, and then the mixed solution is placed in the refrigerator at 4° C. for 24 h to precipitate a crystal. The mixed solution with the crystal is centrifuged with a speed of 3000 r/min for 2 min, with a total of 3 centrifugations, until no more crystals precipitate. The obtained crystals are dried at 100° C. for 2 h to obtain dried crystals for later use. When using the dried crystals, physiological saline is added to prepare a MSU crystal suspension with a concentration of 25 mg/mL.

### 3. Index Detection

3.1 Detection of pain sense of acute gouty arthritis in rats

A weight distribution of rat hind paws is measured by using the bipedal balance pain meter. During testing, the rats stand indoors with their front paws lying on an inclined surface, while their rear paws stand on two different converter planes. Wait for the rats to be in a quiet state and perform a weight distribution testing, with a detection duration set to 9 seconds. The weight of injected right hind limb and the weight of the left side are measured. Each rat is measured three times. The pain sense is indicated by the weigh difference between the right and left of rats.

3.2 Observation of joints morphology of acute gouty arthritis rats

After constructing model for 12 h, the joints of the rats are photographed to observe the swelling degree of the joints.

3.3 Detection of serum inflammatory factors in acute gouty arthritis rats

The inflammatory cytokines of TNF-α and IL-1β in the serum of the rats are measured by using an enzyme-linked immunosorbent assay, an acute gouty arthritis model and the effect of the ethyl acetate extract of the Ligusticum chuanxiong on inflammatory factors are evaluated.

3.3 Joint synovitis pathological examination in acute gouty arthritis rats

Ankle joint tissues of the rats are cleaned, and the ankle joint tissues are placed in a sufficient 4% paraformaldehyde fixed solution for fixing at least 24 h. The ankle joint tissues are decalcified with a decalcification solution, fresh decalcification solution is replaced every three days during decalcification, until the tissues become soft, and a pin can penetrate into the tissues smoothly, when hand touches bone tissues with elasticity and can bend easily, it is judged as a decalcification end point, thus soft ankle joint tissues are obtained. A paraffin embedding is performed on the soft ankle joint tissues to obtain an ankle joint tissue paraffin, the ankle joint tissue paraffin is sliced with the slicing machine to obtain paraffin sections. The paraffin sections are individually dewaxed with xylene I and xylene II for 15 min to obtain dewaxed sections, and the dewaxed sections are individually rehydrated with an ethanol gradient from high to low to obtain rehydrated sections. The rehydrated sections are placed into a hematoxylin solution for 3-5 min, followed by wash with purified water, differentiation with a differentiation solution, rinse with pure water, staining with eosin for 4 min, returning to blue with a bluing reagent, dehydration with the ethanol gradient from high to low after rinsing, transparency with xylene, and sealing with a neutral balsam to obtain sealed sections, and pathological changes of joints in the rats are observed under a light microscope. Microscopic examination, and image acquisition and analysis are performed.

3.4 A statistical method

All experiments are performed individually, and each experiment is repeated at least three times. Data is represented by average ±standard deviation. A SPSS 20.0 software and a GraphPad Prism 9.0 software are adopted to analyze the data, the one-way analysis of variance is used for inter group analysis, and a Tukey test is used for paired comparison. P<0.05 has statistical significance.

### 4. Results and Analysis

4.1 The effect of the ethyl acetate extract of the Ligusticum chuanxiong on the pain sense in acute gouty arthritis rats

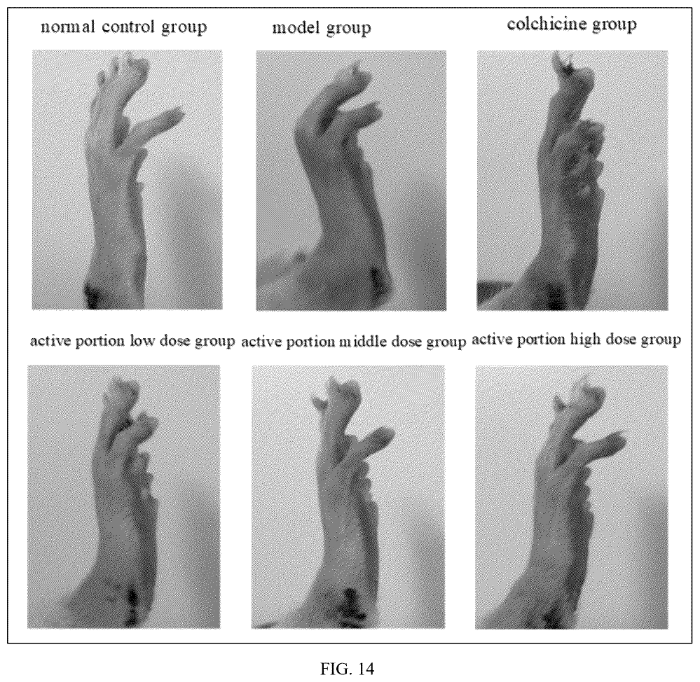

The weight distribution difference of rats in each group is statistically analyzed by bipedal balance pain meter. Results are shown in FIG. 13, a difference of weight distribution in the normal control group is smaller, compared to the normal control group, a difference of weight distribution in the model group is larger, which indicates that pain induced by right foot modeling in the rats leads to an increase in left foot load, after intervention with the ethyl acetate extract of the Ligusticum chuanxiong, a difference of weight distribution between the two feet of the rats is decreased, based on this result, it is speculated that the ethyl acetate extract of Ligusticum chuanxiong has a certain effect of relieving pain.

4.2 The effect of the ethyl acetate extract of Ligusticum chuanxiong on the joint morphology in acute gouty arthritis rats

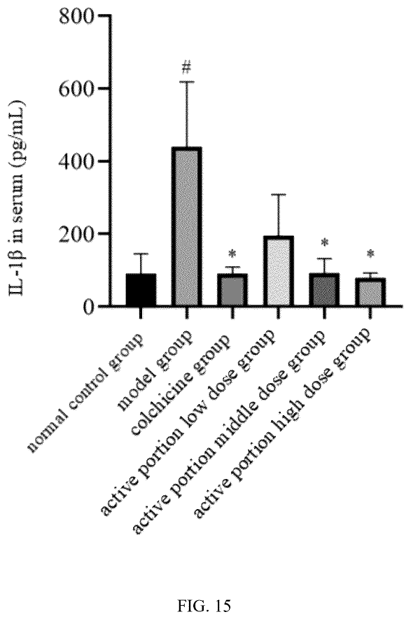

As shown in FIG. 14, compared to the normal control group, paws of the rats in the model group are significantly red and swollen, and the joints are curled up and stiff. Compared to the model group, the ethyl acetate extract of Ligusticum chuanxiong significantly improves redness, swelling, and thickening.

4.3 The effect of the ethyl acetate extract of Ligusticum chuanxiong on level of serum IL-1β and TNF-α in acute gouty arthritis rats

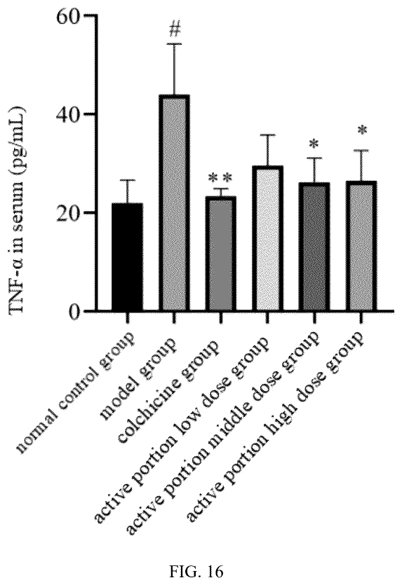

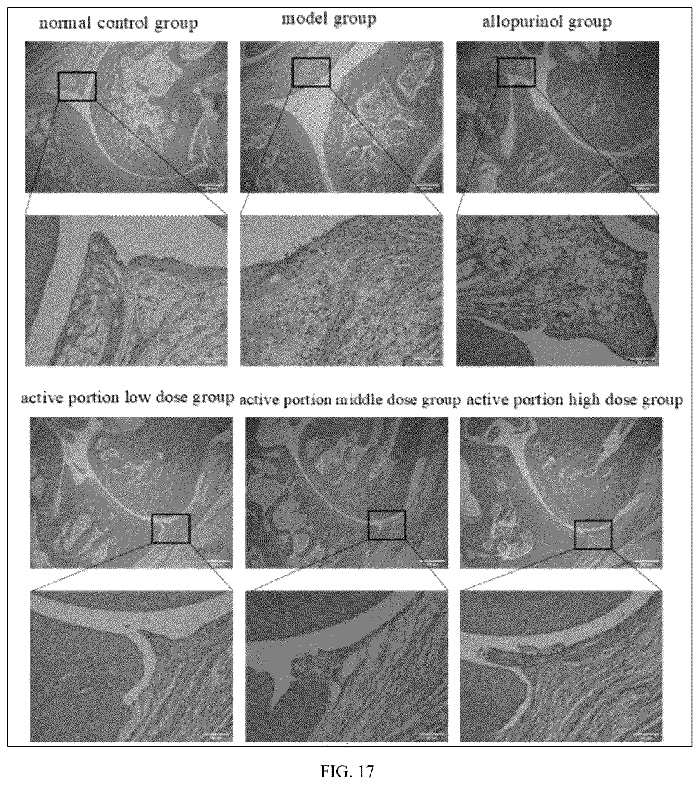

As shown in FIGS. 15 and 16, compared to the normal control group, the inflammatory factor of IL-1β and TNF-α in the model group increases significantly (P<0.05). Compared to the model group, after administration of the ethyl acetate extract of Ligusticum chuanxiong, the IL-1β level and the TNF-α level are reduced obviously. The results demonstrate that the anti-inflammatory effect of ethyl acetate extract of Ligusticum chuanxiong in acute gouty arthritis rats.

4.4 The effect of the ethyl acetate extract of Ligusticum chuanxiong on synovitis pathological of ankle joints in acute gouty arthritis rats

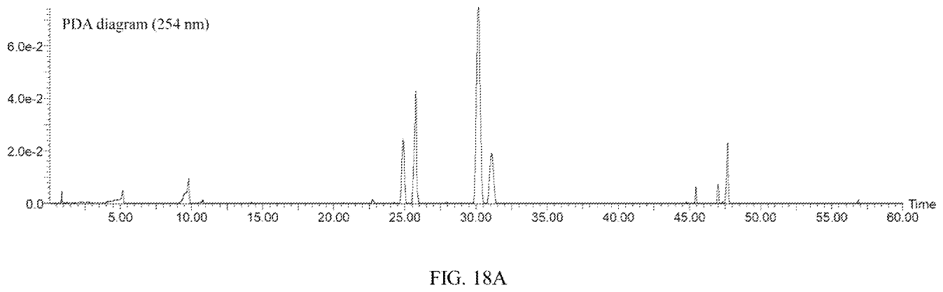

As shown in FIG. 17, synovium of the joints and surrounding tissue cells of the rats in the normal control group are normal and neatly arranged. An arrangement of cells in the synovial tissue of the model group is disorderly, accompanied by inflammatory infiltration. After administration of the ethyl acetate extract of Ligusticum chuanxiong, the inflammatory infiltration in synovium and the arrangement of cells are significantly improved. The results showed that the ethyl acetate extract of Ligusticum chuanxiong obviously improves synovitis pathological of ankle joints in acute gouty arthritis rats.

4.5 A statistical method

All experiments are performed individually, and each experiment is repeated at least three times. Data is represented by average ±standard deviation. A SPSS 20.0 software and a GraphPad Prism 9.0 software are adopted to analyze the data, the one-way analysis of variance is used for inter group analysis, and a Tukey test is used for paired comparison. P<0.05 has statistical significance.

### Embodiment 5

The embodiment provides a component characterization of the ethyl acetate extract of Ligusticum chuanxiong based on an ultra-high performance liquid chromatography-quadrupole time-of-flight mass spectrometry/mass spectrometry (UPLC-Q-TOF MS/MS) technology.

### 1. Experimental Method

1.1 Preparation of a test sample

Appropriate amount of the ethyl acetate extract of Ligusticum chuanxiong is taken and accurately weighed. Methanol is added to prepare a test sample solution with a concentration of 50 mg/mL, the test sample solution is filtered through a microporous filter membrane of 0.22 m for later use.

1.2 Chromatographic conditions

Mobile phase: 0.1% of formic acid water (A)-acetonitrile (B); gradient elution procedure: 0-25 min, 10%-35% B, 25-32 min, 35%-36% B, 32-40 min, 36%-40% B, 40-45 min, 40%-55% B, 51-60 min, and 65%-80% B; flow rate: 0.3 millimeters per minute (mL/min); sample introduction volume: 3 μL; detection wavelength: 254 nm; and temperature of column: 30° C.

1.3 Mass spectrometry analysis conditions

A waters mass spectrometer (SYNAPT G2-Si Q-TOF); positive electrospray ion source (ESI+); capillary voltage: 3.0 kilovoltages (kV); source temperature: 100° C.; desolvation temperature: 350° C.; desolvation gas flow: 800 liters per hour (L/h); and conical gas flow: 50 L/h.

### 2. Results and Analysis

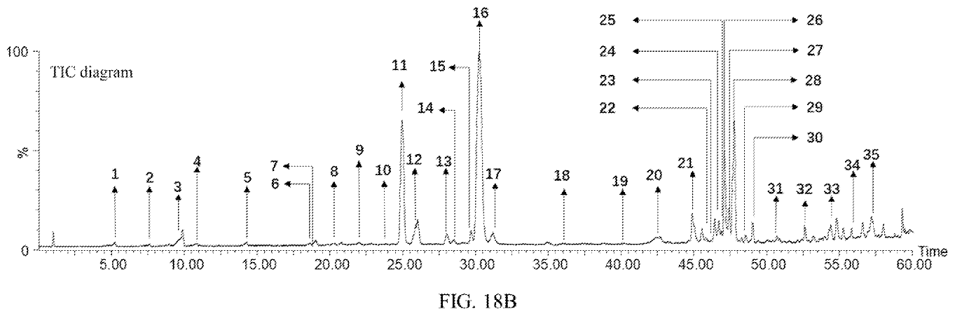

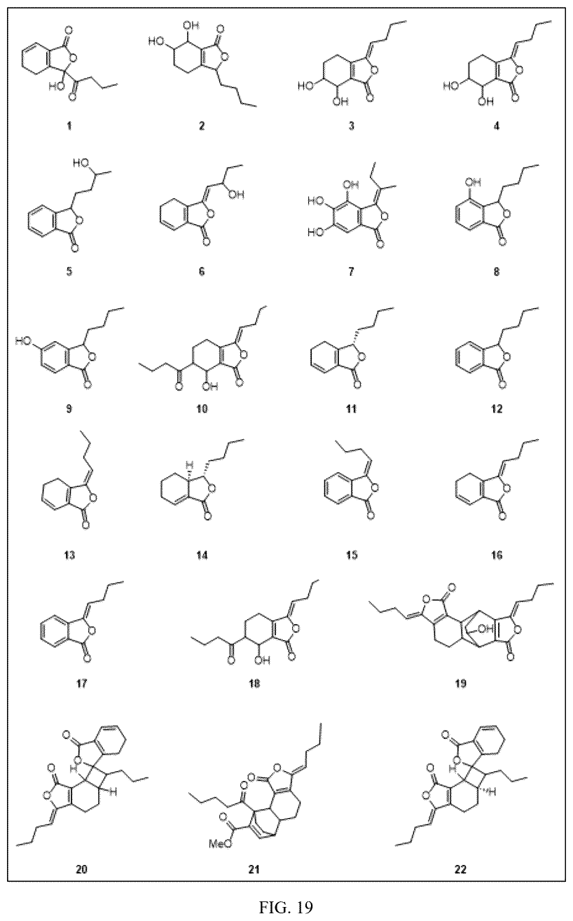

According to the above conditions, an ultra-high performance liquid chromatography (UPLC) chromatogram and a total ion chromatography (TIC) diagram of a positive ion mode are obtained as shown in FIG. 18A and FIG. 18B. Through reviewing documents, a database of chemical components of the Ligusticum chuanxiong is constructed, and components in the ethyl acetate extract are characterized by combining with a database of chemical components of common traditional Chinese medicine, and comparing to mass spectrometry information of the Ligusticum chuanxiong, a retention time, a molecular formula, and a fragment ion of each component are shown in Table 1, and inferred structures are shown in FIG. 19.

TABLE 1
Chromatographic and mass spectrometry data of main components of the ethyl acetate extract of Ligusticum chuanxiong

| | | | Quasi-molecular | | | |
|---|---|---|---|---|---|---|
| | | | ion (mass- | | | |
| | Retention | | to-charge ratio | Adduct | | |
| Order | time | | abbreviated | ion | | Molecular |
| number | (min) | Name of compound | as m/z) | form | Fragment ion (m/z) | formula |
| 1 | 5.23 | senkyunolide D | 223.0576 | +H | 223.0576[M + H]⁺, | C₁₂H₁₄O₄ |
| | | | | | 177.0521[M + H—H₂O—C₂H₄]⁺, | |
| | | | | | 159.1167[M + H—2H₂O—C₂H₄]⁺, | |
| | | | | | 135.0432[M + H—H₂O—C₄H₆O]⁺, | |
| | | | | | 117.0672[M + H—2H₂O—C₄H₆O]⁺ | |
| 2 | 7.60 | senkyunolide | 249.1108 | +Na | 249.1108[M + Na]⁺, | C₁₂H₁₈O₄ |
| | | J/N | | | 209.1153[M + H—H₂O]⁺, | |
| | | | | | 191.1048[M + H—H₂O—CO]⁺, | |
| | | | | | 153.0540[M + H—H₂O—C₄H₈]⁺ | |
| 3 | 9.59 | senkyunolide I | 247.0884 | +Na | 247.0884[M + Na]⁺, | C₁₂H₁₆O₄ |
| | | | | | 207.0993[M + H—H₂O]⁺, | |
| | | | | | 189.0876[M + H—2H₂O]⁺, | |
| | | | | | 179.1038[M + H—H₂O—CO]⁺, | |
| | | | | | 165.9806[M + H—H₂O—C₃H₆]⁺, | |
| | | | | | 161.0939[M + H—2H₂O—CO]⁺, | |
| | | | | | 147.0783[M + H—2H₂O—C₃H₆]⁺, | |
| | | | | | 145.0988[M + H—2H₂O—CO₂]⁺, | |
| | | | | | 133.1002[M + H—2H₂O—CO—C₂H₄]⁺ | |
| 4 | 10.85 | senkyunolide H | 247.0884 | +Na | 247.0884[M + Na]⁺, | C₁₂H₁₆O₄ |
| | | | | | 207.0993[M + H—H₂O]⁺, | |
| | | | | | 189.0876[M + H—2H₂O]⁺, | |
| | | | | | 179.1038[M + H—H₂O—CO]⁺, | |
| | | | | | 165[M + H—H₂O—C₃H₆]⁺, | |
| | | | | | 161.0939[M + H—2H₂O—CO]⁺, | |
| | | | | | 147[M + H—2H₂O—C₃H₆]⁺, | |
| | | | | | 133.0649[M + H—2H₂O—CO—C₂H₄]⁺ | |
| 5 | 14.24 | 3-(3-hydroxybutyl)- | 207.0993 | +H | 207.0993[M + H]⁺, | C₁₂H₁₄O₃ |
| | | 1(3H)isobenzofuran- | | | 189.0876[M + H—H₂O]⁺, | |
| | | one | | | 179[M + H—CO]⁺, | |
| | | | | | 171.0774[M + H—H₂O—CO]⁺, | |
| | | | | | 133[M + H—2CO—H₂O]⁺ | |
| 6 | 18.54 | senkyunolide F | 207.0993 | +H | 207.0993[M + H]⁺, | C₁₂H₁₄O₃ |
| | | | | | 189.0876[M + H—H₂O]⁺, | |
| | | | | | 171.0774[M + H—2H₂O]⁺, | |
| | | | | | 147.0412[M + H—H₂O—C₃H₆]⁺, | |
| | | | | | 133.0246[M + H—H₂O—C₄H₈]⁺ | |
| 7 | 19.01 | (E)-3-butylidene- | 237.0528 | +H | 237.0528[M + H]⁺, | C₁₂H₁₂O₅ |
| | | 4,5,6-trihydroxy-1 | | | 219.0448[M + H—H₂O]⁺, | |
| | | (3H) Isobenzof-uranone | | | 191.1048[M + H—H₂O—C₂H₄]⁺, | |
| | | | | | 163.0318 [M + H—H₂O—CO]⁺ | |
| 8 | 20.24 | 4-hydroxy-3- | 207.0993 | +H | 207.0993[M + H]⁺, | C₁₂H₁₄O₃ |
| | | butylphthalide | | | 189.0876[M + H—2H₂O]⁺, | |
| | | | | | 161.0939[M + H—H₂O—CO]⁺, | |
| | | | | | 171.0774[M + H—2H₂O]⁺, | |
| | | | | | 151.0345[M + H—C₄H₈]⁺ | |
| 9 | 22.20 | 5-hydroxy-3- | 207.0993 | +H | 207.0993[M + H]⁺, | C₁₂H₁₄O₃ |
| | | butylphthalide | | | 189.0876[M + H—2H₂O]⁺, | |
| | | | | | 161.0939[M + H—H₂O—CO]⁺, | |
| | | | | | 171.0774[M + H—2H₂O]⁺, | |
| | | | | | 151.0345[M + H—C₄H₈]⁺ | |
| 10 | 23.80 | senkyunolide | 279.1581 | +H | 279.1581[M + H]⁺, | C₁₆H₂₂O₄ |
| | | M/Q | | | 233.1488[M + H—H₂O—CO]⁺, | |
| | | | | | 191.1408[M + H—H₂O—C₄H₆O]⁺, | |
| | | | | | 177.0872[M + H—C₄H₈O—C₂H₆]⁺, | |
| | | | | | 145.0620[M + H—2H₂O—C₄H₆O—CO]⁺ | |
| 11 | 24.96 | senkyunolide A | 193.1205 | +H | 193.1205[M + H]⁺, | C₁₂H₁₆O₂ |
| | | | | | 175.1110[M + H—H₂O]⁺, | |
| | | | | | 147.0592[M + H—CO]⁺, | |
| | | | | | 137.0592[M + H—C₄H₈]⁺, | |
| | | | | | 119.0848[M + H—CO—C₂H₄]⁺, | |
| | | | | | 105.0686[M + H—CO—C₂H₄—CH₂]⁺ | |
| 12 | 25.97 | 3-butylphthalide | 191.1048 | +H | 191.1048[M + H]⁺, | C₁₂H₁₄O₂ |
| | | | | | 173.0941[M + H—H₂O]⁺, | |
| | | | | | 145.0988[M + H—CO—H₂O]⁺, | |
| | | | | | 135.0420[M + H—C₄H₈]⁺, | |
| | | | | | 117.0679[M + H—CO—H₂O—C₂H₄]⁺, | |
| | | | | | 91.0524[M + H—C₄H₈—CO₂]⁺ | |
| 13 | 27.99 | E-ligustilide | 191.1048 | +H | 191.1048[M + H]⁺, | C₁₂H₁₄O₂ |
| | | | | | 173.0941[M + H—H₂O]⁺, | |
| | | | | | 163[M + H—CO]⁺, | |
| | | | | | 155.0814[M + H—H₂O—H₂O]⁺, | |
| | | | | | 149.065[M + H—C3H6]⁺, | |
| | | | | | 145.0988[M + H—CO—H₂O]⁺, | |
| | | | | | 117.0679[M + H—CO—H₂O—C₂H₄]⁺ | |
| 14 | 28.56 | neocnidilide/ | 195.1375 | +H | 195.1375[M + H]⁺, | C₁₂H₁₈O₂ |
| | | c-nidilide | | | 177[M + H—H₂O]⁺, | |
| | | | | | 149.1298[M + H—H₂O—CO]⁺, | |
| | | | | | 121[M + H—H₂O—CO—C₂H₄]⁺, | |
| | | | | | 107[M + H—H₂O—CO—C₂H₄—CH₂]⁺ | |
| 15 | 29.15 | E-butylidenepht- | 189.0876 | +H | 189.0876[M + H]⁺, | C₁₂H₁₂O₂ |
| | | halide | | | 171.0774[M + H—H₂O]⁺, | |
| | | | | | 153.0648[M + H—2H₂O]⁺, | |
| | | | | | 143.0805[M + H—H₂O—CO]⁺, | |
| | | | | | 128.0599[M + H—H₂O—CO—CH₃]⁺, | |
| | | | | | 117.0679[M + H—CO—C₂H₄]⁺ | |
| 16 | 30.25 | Z-ligustilide | 191.1048 | +H | 191.1048[M + H]⁺, | C₁₂H₁₄O₂ |
| | | | | | 173.0941[M + H—H₂O]⁺, | |
| | | | | | 163.1067[M + H—CO]⁺, | |
| | | | | | 155.0814[M + H—H₂O—H₂O]⁺, | |
| | | | | | 149.065[M + H—C3H6]⁺, | |
| | | | | | 145.0988[M + H—CO—H₂O]⁺, | |
| | | | | | 117.0679[M + H—CO—H₂O—C₂H₄]⁺ | |
| 17 | 31.31 | Z-butylidenepht- | 189.0876 | +H | 189.0876[M + H]⁺, | C₁₂H₁₂O₂ |
| | | halide | | | 171.0774[M + H—H₂O]⁺, | |
| | | | | | 153.0648[M + H—2H₂O]⁺, | |
| | | | | | 128.9461[M + H—H₂O—CO—CH₃]⁺, | |
| | | | | | 115[M + H—H₂O—CO—C₂H₄]⁺, | |
| | | | | | 117.0679[M + H—CO—C₂H₄]⁺ | |
| 18 | 36.06 | senkyunolide | 279.1581 | +H | 279.1581[M + H]⁺, | C₁₆H₂₂O₄ |
| | | M/Q | | | 233.1488[M + H—H₂O—CO]⁺, | |
| | | | | | 191.1408[M + H—H₂O—C₄H₆O]⁺, | |
| | | | | | 177.0872[M + H—C₄H₈O—C₂H₆]⁺, | |
| | | | | | 145.0620[M + H—2H₂O—C₄H₆O—CO]⁺, | |
| | | | | | 143.0805[M + H—H2O—CO]⁺ | |
| 19 | 40.38 | Chuanxiongno- | 397.1968 | +H | 397.1968[M + H]⁺, | C₂₄H₂₈O₅ |
| | | lide A/B | | | 191.1048[C₁₂H₁₅O₂]⁺, | |
| | | | | | 173.0941[C₁₂H₁₅O₂—H₂O]⁺, | |
| | | | | | 207.0993[397.1968—C12H14O2]⁺, | |
| | | | | | 145.0988[C₁₂H₁₅O₂—CO—H₂O]⁺ | |
| 20 | 42.49 | riligustilide | 399.2187 | +H, | 399.2187[M + NH₄]⁺, | C₂₄H₂₈O₄ |
| | | | | +NH₄ | 381.1979[M + H]⁺, | |
| | | | | | 191.1048[C₁₂H₁₅O₂]⁺, | |
| | | | | | 173.0959[C₁₂H₁₅O₂—H₂O]⁺ | |
| 21 | 45.0 | (Z)-3,8-dihydro- | 383.2130 | +H | 383.2130[M + H]⁺, 365[M + | C₂₄H₃OO₄ |
| | | 6,6′,7,3′a- | | | H—H₂O]⁺, 355[M + H—CO]⁺, | |
| | | diligustilide | | | 193.1205[M + H—C₁₂H₁₄O₂]⁺, | |
| | | | | | 191.1048[M + H—C₁₂H₁₆O₂]⁺ | |
| 22 | 45.47 | angelicolide | 381.1979 | +H | 381.1979[M + H]⁺, | C₂₄H₂₈O₄ |
| | | | | | 191.1048[M + H—C₁₂H₁₄O₂]⁺, | |
| | | | | | 173.0959[M + H—C₁₂H₁₄O₂—H₂O]⁺, | |
| | | | | | 145.1014[M + H—C₁₂H₁₄O₂—CO—H₂O]⁺ | |
| 23 | 45.554 | 3,8-dihydro- | 383.2215 | +H | 383[M + H]⁺, 365.2024[M + | C₂₄H₃₀O₄ |
| | | levistilide A | | | H—H₂O]⁺, 355[M + H—CO]⁺, | |
| | | | | | 193.1205[M + H—C₁₂H₁₄O₂]⁺, | |
| | | | | | 191.1048[M + H—C₁₂H₁₆O₂]⁺ | |
| 24 | 46.42 | senkyunolide P | 383.2212 | +H | 383[M + H]⁺, 365.2024[M + | C₂₄H₃₀O₄ |
| | | | | | H—H₂O]⁺, 355[M + H—CO]⁺, | |
| | | | | | 193.1205[M + H—C₁₂H₁₄O₂]⁺, | |
| | | | | | 191.1048[M + H—C₁₂H₁₆O₂]⁺ | |
| 25 | 46.72 | 3,8-dihydro- | 385.2332 | +H | 385.2332[M + H]⁺, | C₂₄H₃₂O₄ |
| | | diligustilide/c- | | | 367.2264[M + H—H₂O]⁺, | |
| | | huanxiongdiol-ide A | | | 349.2152[M + H—H₂O]⁺, | |
| | | | | | 193.1205[M + H—C₁₂H₁₆O₂]⁺ | |
| 26 | 47.10 | methyl ester | 435.2049 | +Na, | 435.2049[M + Na]⁺, | C₂₅H₃₂O₅ |
| | | derived from | | +H | 413.2226[M + H]⁺, | |
| | | angeolide | | | 191.1048[M + H—C₁₃H₁₈O₃]⁺, | |
| | | | | | 173.0941[M + H—C₁₃H₁₉O₃—H₂O]⁺, | |
| | | | | | 145.0988[M + H—C₁₃H₁₉O₃—CO—H₂O]⁺ | |
| 27 | 47.43 | wallichilide | 413.2226 | +H | 435.2049[M + Na]⁺, | C₂₅H₃₂O₅ |
| | | | | | 413.2226[M + H]⁺, | |
| | | | | | 191.1048[M + H—C₁₃H₁₈O₃]⁺, | |
| | | | | | 173.0941[M + H—C₁₃H₁₉O₃—H₂O]⁺, | |
| | | | | | 145.0988[M + H—C₁₃H₁₉O₃—CO—H₂O]⁺, | |
| | | | | | 117.0679[M + H—C₁₃H₁₉O₃—CO—H₂O—C₂H₄]⁺ | |
| 28 | 47.43 | levistolide B | 381.1979 | +H | 381.1979[M + H]⁺, | C₂₄H₂₈O₄ |
| | | | | | 335.1993[M + H—H₂O—CO]⁺, | |
| | | | | | 191.1048[M + H—C₁₂H₁₄O₂]⁺, | |
| | | | | | 173.0959[M + H—C₁₂H₁₄O₂—H₂O]⁺ | |
| 29 | 48.29 | senkyunolide O | 381.1979 | +H | 381.1979[M + H]⁺, | C₂₄H₂₈O₄ |
| | | | | | 191.1048[M + H—C₁₂H₁₄O₂]⁺, | |
| | | | | | 173.0941[M + H—C₁₂H₁₄O₂—H₂O]⁺, | |
| | | | | | 163[M + H—C₁₂H₁₄O₂—CO]⁺, | |
| | | | | | 155[M + H—C₁₂H₁₄O₂—2H₂O]⁺ | |
| 30 | 49.04 | levistolide A | 381.1979 | +H | 381.1979[M + H]⁺, | C₂₄H₂₈O₄ |
| | | | | | 191.1048[M + H—C₁₂H₁₄O₂]⁺, | |
| | | | | | 173.0941[M + H—C₁₂H₁₄O₂—H₂O]⁺ | |
| 31 | 50.70 | (3Z)-(3aR,6S,3′R,8S)- | 381.1979 | +H | 381.1979[M + H]⁺, | C₂₄H₂₈O₄ |
| | | 3a.8′,6.3′- | | | 335.2805[M + H—H₂O—CO]⁺, | |
| | | diligustilide | | | 191.1048[M + H—C₁₂H₁₄O₂]⁺, | |
| | | | | | 149.1298[M + H—C₁₂H₁₄O₂—C₃H₆]⁺ | |
| 32 | 52.61 | tokinolide A | 381.1979 | +H | 381.1979[M + H]⁺, | C₂₄H₂₈O₄ |
| | | | | | 191.1048[[M + H—C₁₂H₁₄O₂]⁺, | |
| | | | | | 173[M + H—C₁₂H₁₄O₂—H₂O]⁺ | |
| 33 | 54.42 | chaxiongnolide F | 383.2215 | +H | 383.2215[M + H]⁺, 365[M + H—H₂O]⁺, | C₂₄H₃₀O₄ |
| | | | | | 191.1048[C₁₂H₁₅O₂]⁺, | |
| | | | | | 225.1579[M + H—C₆H₈O₂—H₂O—CO]⁺, | |
| | | | | | 193.1205[M + H—C₁₂H₁₄O₂]⁺ | |
| 34 | 55.26 | tokiaerialide | 381.1971 | +H | 381.1979[M + H]⁺, | C₂₄H₂₈O₄ |
| | | | | | 191.1048[C₁₂H₁₅O₂]⁺, | |
| | | | | | 173.0941 [C₁₂H₁₅O₂—H₂O]⁺ | |
| 35 | 57.18 | neodiligustilide | 381.1973 | +H | 381.1979[M + H]⁺, | C₂₄H₂₈O₄ |
| | | | | | 191.1048[M + H—C₁₂H₁₄O₂]⁺, | |
| | | | | | 173.0959[M + H—C₁₂H₁₄O₂—H₂O]⁺, | |
| | | | | | 145.0988[M + H—C₁₂H₁₄O₂—CO—H₂O]⁺ | |

As mentioned above, the disclosure can be effectively implemented. The above embodiments merely describe the embodiments of the disclosure and do not limit a scope of the disclosure. Without departing from a design spirit of the disclosure, all changes and improvements made by those skilled in the art to technical solutions of the disclosure should fall within a scope of protection determined by the disclosure.

What is claimed is:

1. An application method of an active portion of Ligusticum chuanxiong, comprising:
applying the active portion of the Ligusticum chuanxiong to prepare an anti-gout drug.

2. The application method as claimed in claim 1, wherein the active portion of the Ligusticum chuanxiong is an ethyl acetate extract.

3. The application method as claimed in claim 2, wherein the ethyl acetate extract is obtained by using ethyl acetate to extract the Ligusticum chuanxiong.

4. The application method as claimed in claim 2, wherein the ethyl acetate extract comprises a polar lipophilic component.

5. The application method as claimed in claim 4, wherein the polar lipophilic component is a phthalide compound.

6. The application method as claimed in claim 2, wherein a half maximal inhibitory concentration (IC₅₀) value of the ethyl acetate extract for inhabiting xanthine oxidase (XOD) is in a range of 18.06-18.44 micrograms per milliliter (μg/mL).

7. An anti-gout drug, comprising an ethyl acetate extract of Ligusticum chuanxiong.

8. The anti-gout drug as claimed in claim 7, wherein the anti-gout health product is prepared into a convenient dosage form for use selected from the group consisting of a tablet, a pill, a paste, a spray, and a capsule.

9. An anti-gout health product, comprising an ethyl acetate extract of Ligusticum chuanxiong.

10. The anti-gout health product as claimed in claim 9, wherein the anti-gout health product is prepared into a convenient dosage form for use selected from the group consisting of a tablet, a pill, a paste, a spray, and a capsule.
